(19) United States
(12) Patent Application Publication
(10) Pub. No.: US 2023/0115171 A1
(43) Pub. Date: Apr. 13, 2023

(54) SYSTEM AND METHOD FOR GENERATING CUSTOMIZABLE MOLECULAR STRUCTURES FOR DRUG DISCOVERY

(71) Applicant: Innoplexus AG, Eschborn (DE)

(72) Inventors: Vivek Singh, Pune (IN); Bibhash Mitra, Burdwan (IN); Ashwin Rathod, Nanded (IN); Rohit Yadav, Indore (IN)

(21) Appl. No.: 17/497,190

(22) Filed: Oct. 8, 2021

(51) Int. Cl. G16B 15/30; G06N 3/08; G06N 3/04; G16B 5/20; G16C 20/50; G16C 20/70

(52) U.S. Cl. CPC G16B 15/30; G06N 3/08; G06N 3/0427; G16B 5/20; G16C 20/50; G16C 20/70

(57) ABSTRACT

A system and method for generating customizable molecular structures for drug discovery. The system includes a processor communicably coupled to a memory and executes a deep neural network based molecular encoding model. The processor receives input datasets of drug-like molecules from private and public databases and are employed as training dataset. The processor further executes a plurality of deep generative models configured to receive input data relating to small molecules which includes desirable molecules and undesirable molecules. The plurality of deep generative models generates molecular structures like the input desirable molecules. The deep neural network based molecular encoding model is configured to map similarities between the molecular structures generated. The deep neural network based molecular encoding model computes intra-model and inter-model distances. Further, the deep neural network based molecular encoding model samples the molecular structures generated from the plurality of deep generative models to obtain desired molecular structure.

### TECHNICAL FIELD

The present disclosure relates generally to drug discovery; and more specifically, to system and method for generating customizable molecular structures for drug discovery.

### BACKGROUND

Conventionally, developing a new drug has been a complex, costly, and time-consuming process. Additionally, the process requires about 12-15 years of time, and the cost may go up to 2.8 billion dollars. Moreover, a drug discovery program is initiated in order to tackle a disease or clinical condition without the availability of suitable medical products. Furthermore, it may take several years to build up a body of supporting evidence before selecting a target for a costly drug discovery program. Additionally, once a target has been chosen, the pharmaceutical industry and more recently some academic centres have streamlined a number of early processes to identify molecules which possess suitable characteristics to make acceptable drugs. Notably, many drugs are small molecules, and discovery or designing of such small molecules is central to any drug discovery endeavour. Furthermore, a small molecule needs to satisfy several physicochemical and biological criteria for it to be a potential drug-like molecule. Moreover, these criteria include strong binding affinity to the drug target of interest, novelty, easy to chemically synthesize to facilitate various assay, satisfy drug-like properties such as Lipinski's rules and optimal ADMET (Absorption, Distribution, Metabolism, Excretion, Toxicity) characteristics.

Notably, the drug discovery process along with the generation of right molecular structure is traditionally time-consuming, costly, and often incomplete. Additionally, the rate of success in the area of getting desirable molecules that succeed in a drug discovery pipeline is very limited. Moreover, another main challenge in small molecule drug discovery is finding novel chemical compounds with the desirable properties.

Typically, existing solutions includes survey of scientific literature and patents to identify promising molecules and chemical moieties around which molecules with desirable properties can be designed. Additionally, use of chemical knowledge bases and chemical structure drawing tools for designing of molecules with desirable properties based on the existing knowledge bases are being used. Moreover, performing series of in silico high-throughput assays with various endpoints to predict whether the designed molecules possess the desired characteristics are also being tried. Furthermore, some solutions involve performing molecular docking-based analysis and/or biological assays with purified proteins to assess the binding of the designed molecules to the protein of interest. However, the existing methods suffer from several limitations. Notably, lack of novelty in molecular structures are seen as the molecules are derived primarily by making small alterations to already existing molecules. Moreover, even if novel molecular structures are created by using desirable substructures of existing molecules, factors such as stability, ease of synthesis, and the likes are compromised.

Therefore, in light of the foregoing discussion, there exists a need to overcome the aforementioned drawbacks associated with the generation of customizable molecular structures in the process of drug discovery.

### SUMMARY

The present disclosure seeks to provide a system for generating customizable molecular structures for drug discovery. The present disclosure also seeks to provide a method for generating customizable molecular structures for drug discovery. An aim of the present disclosure is to provide a solution that overcomes at least partially the problems encountered in prior art.

In one aspect, the present disclosure provides a system for generating customizable molecular structures for drug discovery, wherein the system comprises a processor communicably coupled to a memory, the processor configured to execute:
a deep neural network based molecular encoding model, wherein the processor receives input datasets of drug-like molecules from private and public databases;
wherein the received input datasets are employed as training data for the deep neural network based molecular encoding model;
a plurality of deep generative models configured to:
   receive input data relating to small molecules; wherein the input data comprises data relating to desirable molecules and undesirable molecules;
   generate molecular structures in accordance with the objective function of the generative model;
wherein the deep neural network based molecular encoding model is further configured to:
   map similarities between the molecular structures generated from the plurality of deep generative models; wherein the molecular structures generated from individual generative models are mapped on a n-dimensional latent space of the deep neural network based molecular encoding model;
   compute intra-model and inter-model distances of the generated molecular structures; and
   sample the molecular structures generated from the plurality of deep generative models to obtain desired molecular structure.

In another aspect, the present disclosure provides a method for generating customizable molecular structures for drug discovery, wherein the method comprises:
training a deep neural network based molecular encoding model, wherein a processor receives input datasets of drug-like molecules from private and public databases;
receiving input data relating to small molecules by a plurality of deep generative models; wherein the input data comprises data relating to desirable molecules and undesirable molecules;
generating molecular structures in accordance with the objective function of the generative model;
mapping similarities between the molecular structures generated from the plurality of deep generative models; wherein the molecular structures generated from the individual generative models are mapped on a n-dimensional latent space of the deep neural network based molecular encoding model;
computing intra-model and inter-model distances of the generated molecular structures; and
sampling the molecular structures generated from the plurality of deep generative models to obtain desired molecular structure.

Embodiments of the present disclosure substantially eliminate or at least partially address the aforementioned problems in the prior art, and allows integration of individual generative models to generate molecular structures that have optimized characteristics.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic is a schematic illustration of a system for generating customizable molecular structures for drug discovery, in accordance with an embodiment of the present disclosure;

FIG. 2 is a schematic illustration depicting a deep generative model for the generation of a molecular structure, in accordance with an exemplary implementation of the present disclosure; and

FIG. 3 is a flowchart depicting steps of a method for generating customizable molecular structures for drug discovery, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, the present disclosure provides a system for generating customizable molecular structures for drug discovery, wherein the system comprises a processor communicably coupled to a memory, the processor configured to execute:
a deep neural network based molecular encoding model, wherein the processor receives input datasets of drug-like molecules from private and public databases;
wherein the received input datasets are employed as training data for the deep neural network based molecular encoding model;
a plurality of deep generative models configured to:
   receive input data relating to small molecules; wherein the input data comprises data relating to desirable molecules and undesirable molecules;
   generate molecular structures in accordance with the objective function of the generative model;

wherein the deep neural network based molecular encoding model is further configured to:
map similarities between the molecular structures generated from the plurality of deep generative models; wherein the molecular structures generated from individual generative models are mapped on a n-dimensional latent space of the deep neural network based molecular encoding model;
   compute intra-model and inter-model distances of the generated molecular structures; and
   sample the molecular structures generated from the plurality of deep generative models to obtain desired molecular structure.

In another aspect, the present disclosure provides a method for generating customizable molecular structures for drug discovery, wherein the method comprises:
training a deep neural network based molecular encoding model, wherein a processor receives input datasets of drug-like molecules from private and public databases;
receiving input data relating to small molecules by a plurality of deep generative models; wherein the input data comprises data relating to desirable molecules and undesirable molecules;
generating molecular structures in accordance with the objective function of the generative model;
mapping similarities between the molecular structures generated from the plurality of deep generative models; wherein the molecular structures generated from the individual generative models are mapped on a n-dimensional latent space of the deep neural network based molecular encoding model;
computing intra-model and inter-model distances of the generated molecular structures; and
sampling the molecular structures generated from the plurality of deep generative models to obtain desired molecular structure.

The system and method of the present disclosure aims to provide generation of customizable molecular structures for drug discovery. Notably, the present disclosure allows integration of individual generative models to generate molecular structures that have optimized characteristics. Additionally, the molecular structures generated are novel with characteristics such as stability, ease of synthesis. Consequently, the present disclosure generates molecular structures that bind to a protein of interest, or, result in a desirable omics (e.g. transcriptomic) signature, and satisfies drug-like properties such as solubility, bioavailability, non-toxicity, and the like.

Pursuant to embodiments of the present disclosure, the system and the method provided herein are for generating customizable molecular structures for drug discovery. Herein, “customizable molecular structures” refers to the molecular structures that can be tailored to a particular need such as binding capability to a protein of interest, is novel and satisfies drug-like properties such as solubility, bioavailability, non-toxicity, and the like. The molecular structures satisfy drug-like properties such as Lipinski's rules and optimal ADMET (Absorption, Distribution, Metabolism, Excretion, Toxicity) characteristics. Herein, “Lipinski's rules” refers to rules of thumb to evaluate drug likeness or to determine if a chemical compound with a certain pharmacological or biological activity has chemical properties and physical properties that would make it a likely orally active drug in humans. Herein, “drug discovery” refers to the process through which potential new medicines are identified. Furthermore, drug discovery involves a wide range of scientific disciplines, including biology, chemistry, and pharmacology.

The system comprises a processor communicably coupled to a memory. Herein, the term “processor” relates to a computational element that is operable to respond to and process instructions that carry out the method. Optionally, the processor includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “processor” may refer to one or more individual processors, processing devices and various elements associated with a processing device that may be shared by other processing devices.

The processor is configured to execute a deep neural network based molecular encoding model, wherein the processor receives input datasets of drug-like molecules from private and public databases; wherein the received input datasets are employed as training data for the deep neural network based molecular encoding model. Herein, “deep neural network based molecular encoding model” refers to an autoencoder framework i.e., a pair of deep neural networks consisting of an encoder and a decoder to encode molecules into a continuous vector representation. Notably, the encoder converts the discrete representation of a molecule into a real-valued continuous vector, and the decoder converts these continuous vectors back to discrete molecular representations. Furthermore, the continuous representations of molecules allow to generate novel chemical structures by performing simple operations, such as decoding random vectors, decoding from nearby points of an encoded molecule, or interpolating between molecules. Moreover, continuous representations also allow the use of powerful gradient-based optimization to efficiently guide the search for optimized functional compounds. Notably, the encoder and decoder generate molecular structures that are distinct from the input molecules but have similar properties. Moreover, the deep neural network based molecular encoding model is trained using drug-like molecules available in ZINC database, and further augmented using molecules from other publicly available sources such as ChEMBL. Notably, the ZINC database is a curated collection of commercially available chemical compounds prepared especially for virtual screening. Moreover, ChEMBL is a manually curated database of bioactive molecules with drug-like properties. Additionally, ChEMBL brings together chemical, bioactivity and genomic data to aid the translation of genomic information into effective new drugs.

Optionally, the processor is configured to represent the input datasets of drug-like molecules as simplified molecular-input line-entry system (SMILES) notation strings. Herein, “SMILES notation string” refers to a format of representing molecular structures in the form of a line notation for describing the structure of chemical species using short ASCII strings. Notably, the input datasets are in SMILES representation. Additionally, the encoder of the deep neural network based molecular encoding model converts each SMILES string into a fixed-dimensional vector and the decoder converts a vector back to a SMILES string. Furthermore, in addition to canonical SMILES as the input dataset molecules, non-canonical SMILES of the same molecules are also used that are obtained from RDKit. Moreover, non-canonical SMILES augment the dataset and create robust molecular encoding.

The processor is configured to execute a plurality of deep generative models. Herein, “deep generative models” refers to deep learning based artificial intelligence models that are used in generating molecular structures for a drug target of interest, or, result in a desirable omics (e.g. transcriptomic) signature. Notably, the plurality of deep generative models may be of varying kind and architectures that generates new molecular structures. Herein, the kind and the architectures of the plurality of deep generative models may be chosen based on the required output. Additionally, the plurality of deep generative models are trained using different types of datasets. Moreover, the plurality of generative models used may be publicly available generative models with interesting approaches for generating desirable molecular structures.

The plurality of deep generative models are configured to receive input data relating to small molecules; wherein the input data comprises data relating to desirable molecules and undesirable molecules. Herein, “input data” refers to relevant dataset used for training the plurality of deep generative models. Additionally, the input data may differ for each of the plurality of deep generative models. Moreover, the input data may include, but not limited to, molecular structures in the form SMILES notation, three-dimensional structures of molecules, target protein, or their complexes, and gene expression or any other types of omics signatures representing a phenotypic endpoint (e.g., cancer). Herein, “desirable molecules” refers to the molecular structures with desired properties and binding affinity towards a protein (target), or, result in a desirable omics (e.g. transcriptomic) signature. Herein, “undesirable molecules” refers to the molecular structures that does not contain the desired properties or binding affinity towards a protein (target).

The plurality of deep generative models are configured to generate molecular structures in accordance with the objective function of the generative model. Notably, the plurality of deep generative models generate output based on the required properties. Herein, the required properties may be absorption, distribution, metabolism, excretion, toxicity, and the likes.

Optionally, the plurality of deep generative models is configured to generate molecular structures for a target of interest. Notably, herein, “target of interest” refers to a target of protein. Additionally, the plurality of deep generative models may be configured to generate molecular structures with optimized ADMET (Absorption, Distribution, Metabolism, Excretion, Toxicity) properties and omics (e.g., transcriptomic) signature corresponding to desirable phenotypic endpoints. Furthermore, the plurality of deep generative models generates molecular structures in SMILES notation that are trained on relevant input dataset for the target of interest.

Optionally, the plurality of deep generative models are configured to optimize the molecular structures based on objective functions of the individual plurality of deep generative models. Notably, the plurality of deep generative models may use different yet complementary approaches of objective functions for optimizing the properties of generated molecules. Additionally, the objective function needs to capture as many desirable traits as possible and balance them to ensure that the plurality of deep generative models focuses on genuinely desirable compounds. Additionally, the plurality of deep generative models uses reward functions only for the deep generative models with reinforcement learning. Herein, reinforcement learning is used to fine-tune the model to preferentially generate target protein specific inhibitors based on objective functions. Herein, objective functions allow to rank the generated molecules based on their binding potential to target protein and additional properties such as novelty, ease of synthesis, etc. Notably, one or more of the plurality of deep generative models may not use reward functions in order to generate molecules but may solely work on the basis of input dataset used for training.

In this regard, the availability of the plurality of deep generative models with their own characteristics and advantages opens a possibility of having an integration (or amalgamation) of these models. Additionally, the plurality of deep generative models together can generate even better and more desirable molecules than any of the individual models by leveraging their individual advantages and overcoming their individual limitations.

In an example, one of the plurality of deep generative model may use input data as synthesizable molecules, common protease inhibitors, known target protein inhibitors, non-protease inhibitors, molecular structure patent data, and three-dimensional structural information. Herein, synthesizable molecules are used to understand the synthesizable molecular space, from which new molecules are generated by the model. Moreover, common protease inhibitors are used to learn key properties required in a molecule to inhibit protease proteins. Furthermore, non-protease inhibitors act as a negative set to help eliminate non-protease binding properties from newly generated molecules. Additionally, molecular structure patent data helps in eliminating molecules that are already patented. Notably, the input data of the molecular structures are expressed in SMILES notation for all downstream processing. Additionally, the input data contains molecular properties such as Quantitative Estimate of Drug-likeness (QED) of molecules, Synthetic Accessibility (SA) of molecules, pIC50 of molecules for target protein, and Medicinal Chemistry Filters (MCFs). Notably, the input data is further processed by removal of mixtures, removal of salts and neutralization of molecules, normalization of specific chemotypes, and analysis/removal of duplicates. Additionally, the deep generative model is trained to understand the latent laws of chemical space and interactions to generate new molecular structures conforming to these latent rules of nature. Moreover, the model further orients the generation of molecules corresponding to the protein target of interest. Notably, the deep generative model learns the characteristics of desirable and undesirable molecules using the input data with positive and negative datasets. Furthermore, the deep generative model is trained to optimize the generation of molecules towards desirable properties, and thus, improving the model at each iteration. Notably, the deep generative model renders reinforcement learning to fine tune itself to preferentially generate target protein specific inhibitors based on objective functions. Herein, objective functions are a set of three Self-Organizing Maps (SOMs) allowing the ranking of generated molecules based on their binding potential to target protein and novelty.

The deep neural network based molecular encoding model is further configured to map similarities between the molecular structures generated from the plurality of deep generative models; wherein the molecular structures generated from individual generative models are mapped on a n-dimensional latent space of the deep neural network based molecular encoding model. Herein, “latent space” refers to an embedding of a set of molecular structures within a manifold in which the molecular structures which resemble each other more closely are positioned closer to one another in the latent space. Furthermore, points in the latent space are decoded into valid SMILES strings that capture the chemical nature of the training data for optimization. Additionally, the encoder-decoder present in the deep neural network based molecular encoding model trained on drug-like molecules from various private and public databases are used for mapping the newly generated molecular structures on the latent space. Moreover, the deep neural network based molecular encoding model learns the features of the newly generated molecules based on the input data provided during training. Herein, mapping features includes but are not limited to SMILES notation of the molecular structures, physicochemical properties (e.g., molecular weight), bioactivity potential, computed ADMET (Absorption, Distribution, Metabolism, Excretion, Toxicity) properties, drug-likeness, synthetic accessibility, and the likes. Notably, the deep neural network based molecular encoding model maps the newly generated molecules based on these features. Furthermore, computation of the similarities between the molecules generated from the plurality of deep generative models is performed by the deep neural network based molecular encoding model.

The deep neural network based molecular encoding model is further configured to compute intra-model and inter-model distances of the generated molecular structures. Notably, the deep neural network based molecular encoding model calculates the intra-model and inter-model distances of the newly generated molecules by the plurality of deep generative models. Additionally, the calculated distances show the likeliness of similarities and dissimilarities between two newly generated molecules. Moreover, the shorter the distances between two newly generated molecules, the more likely they are to have similar properties. Consequently, the bigger the distances between two newly generated molecules, the more likely they are to have dissimilar properties.

Optionally, the inter-model distance is the distance between molecular structures generated by a pair of generative models and intra-model distance is the distance between molecules generated by the same generative model.

The deep neural network based molecular encoding model is further configured to sample the molecular structures generated from the plurality of deep generative models to obtain desired molecular structure. Notably, sampling is performed on the newly generated molecules from the latent space of the deep neural network based molecular encoding model to obtain various types of desirable molecules. Additionally, the desirables molecules include but are not limited to variants of core molecular structures optimized by the individual deep generative models, sampling of molecular structures with differing molecular scaffolds, and sampling of molecular structures sharing characteristics of molecules generated from the plurality of deep generative models.

Optionally, the deep neural network based molecular encoding model is configured to leverage the distances calculated to optimize the molecular structures for desired properties; wherein the desired properties are obtained by sampling the molecular structures that show small inter-model distances between the plurality of deep generative models of interest.

Optionally, the deep neural network based molecular encoding model is configured to obtain diverse molecular structures optimized for different properties of the plurality of deep generative models; wherein diverse molecular structures are obtained by sampling from overlapping regions occupied by molecular structures from the plurality of deep generative models of interest.

In this regard, variants of core molecular structures can also be sampled by the deep neural network based molecular encoding model from this latent space. Additionally, a sample containing molecules from different molecular scaffold can also be obtained for performing exploratory early-stage studies in process of drug discovery. Moreover, the molecules can also be sampled from regions near to the regions occupied by molecules of individual deep generative models, thus, allowing for sampling molecular structures with desirable properties shared from all the individual deep generative models.

The present disclosure also relates to the method as described above. Various embodiments and variants disclosed above apply mutatis mutandis to the method.

Optionally, the method comprises representing the input datasets of drug-like molecules as simplified molecular-input line-entry system (SMILES) notation strings.

Optionally, the method comprises generating molecular structures by the plurality of deep generative models for a target of interest.

Optionally, the method comprises optimizing the molecular structures by the plurality of deep generative models based on objective functions of the individual plurality of deep generative models.

Optionally, the inter-model distance is the distance between molecular structures generated by a pair of generative models and intra-model distance is the distance between molecules generated by the same generative model.

Optionally, the method comprises leveraging the distances calculated by the deep neural network based molecular encoding model to optimize the molecular structures for desired properties; wherein the desired properties are obtained by sampling the molecular structures that show small inter-model distances between the plurality of deep generative models of interest.

Optionally, the method comprises obtaining diverse molecular structures optimized for different properties of the plurality of deep generative models; wherein diverse molecular structures are obtained by sampling from overlapping regions occupied by molecular structures from the plurality of deep generative models of interest.

### DETAILED DESCRIPTION OF THE DRAWINGS

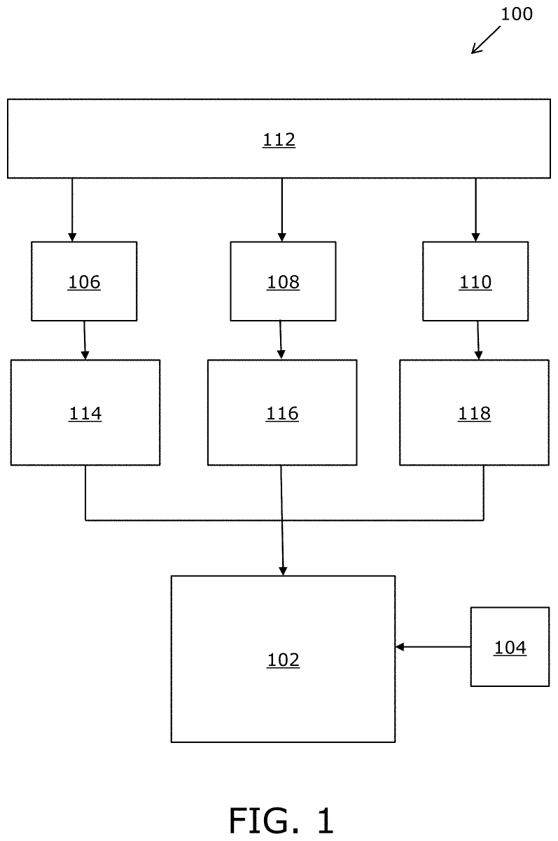

Referring to FIG. 1, illustrated is a schematic illustration of a system 100 for generating customizable molecular structures for drug discovery, in accordance with an embodiment of the present disclosure. The system comprises a processor (not shown) communicably coupled to a memory (not shown). The processor is configured to execute a deep neural network based molecular encoding model 102. The processor receives input datasets 104 of drug-like molecules from private and public databases. The received input datasets 104 are employed as training data for the deep neural network based molecular encoding model 102. The processor is further configured to execute a plurality of deep generative models, such as deep generative models 106, 108, 110. The plurality of deep generative models, such as deep generative models 106, 108, 110 are configured to receive input data 112 relating to small molecules. The input data 112 comprises data relating to desirable molecules and undesirable molecules. The plurality of deep generative models 106, 108, 110 are further configured to generate molecular structures 114, 116, 118 similar to the input desirable molecules. The deep neural network based molecular encoding model 102 is further configured to map similarities between the molecular structures 114, 116, 118 generated from the plurality of deep generative models 106, 108, 110. The molecular structures generated from individual generative models 106, 108, 110 are mapped on a n-dimensional latent space of the deep neural network based molecular encoding model 102. The deep neural network based molecular encoding model 102 is further configured to compute intra-model and inter-model distances of the generated molecular structures 114, 116, 118 and sample the molecular structures from 114, 116, 118 generated from the plurality of deep generative models, such as 106, 108, 110 to obtain desired molecular structure.

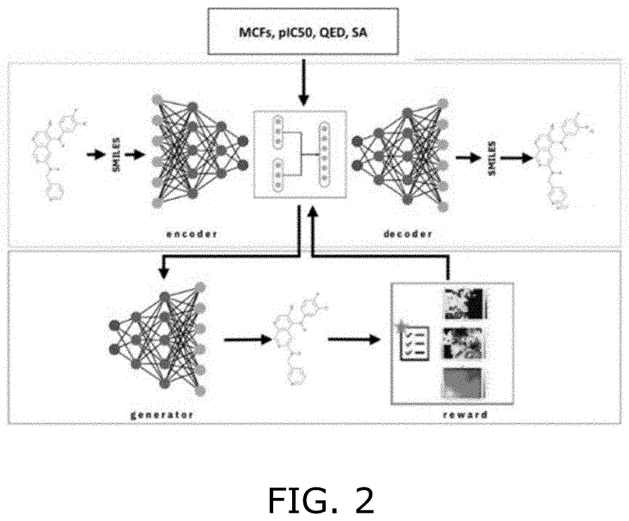

Referring to FIG. 2, illustrated is a schematic illustration depicting a deep generative model for the generation of a molecular structure, in accordance with an exemplary implementation of the present disclosure. Herein, the autoencoder consists of an encoder and a decoder. The encoder converts the discrete representation of a molecule into a real-valued continuous vector, and the decoder converts these continuous vectors back to discrete molecular representations. The encoder and decoder generate molecular structures that are distinct from the input molecules but have similar properties.

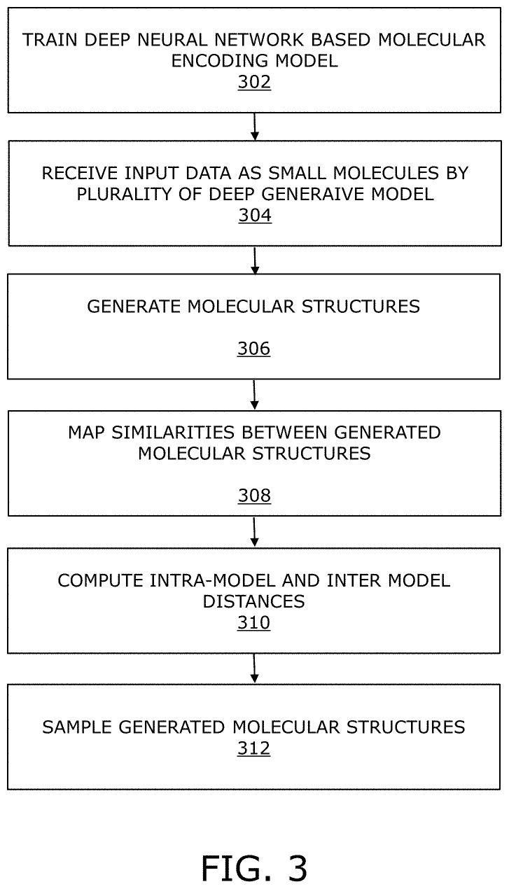

Referring to FIG. 3, illustrated is a flowchart depicting steps of a method for generating customizable molecular structures for drug discovery, in accordance with an embodiment of the present disclosure. At step 302, a deep neural network based molecular encoding model is trained by a processor. The processor receives input datasets of drug-like molecules from private and public databases. At step 304, input data relating to small molecules is received by a plurality of deep generative models. The input data comprises data relating to desirable molecules and undesirable molecules. At step 306, molecular structures in accordance with the objective function of the generative model are generated. At step 308, similarities between the molecular structures generated from the plurality of deep generative models are mapped. The molecular structures generated from the individual generative models are mapped on a n-dimensional latent space of the deep neural network based molecular encoding model. At step 310, intra-model and inter-model distances of the generated molecular structures are calculated. At step 312, the molecular structures generated from the plurality of deep generative models are sampled to obtain desired molecular structure.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A system for generating customizable molecular structures for drug discovery, wherein the system comprises a processor communicably coupled to a memory, the processor configured to execute:
a deep neural network based molecular encoding model, wherein the processor receives input datasets of drug-like molecules from private and public databases; wherein the received input datasets are employed as training data for the deep neural network based molecular encoding model;
a plurality of deep generative models configured to:
receive input data relating to small molecules; wherein the input data comprises data relating to desirable molecules and undesirable molecules;
generate molecular structures in accordance with the objective function of the generative model;
wherein the deep neural network based molecular encoding model is further configured to:
map similarities between the molecular structures generated from the plurality of deep generative models; wherein the molecular structures generated from individual generative models are mapped on a n-dimensional latent space of the deep neural network based molecular encoding model;
compute intra-model and inter-model distances of the generated molecular structures; and
sample the molecular structures generated from the plurality of deep generative models to obtain desired molecular structure.

2. The system of claim 1, wherein the processor is configured to represent the input datasets of drug-like molecules as simplified molecular-input line-entry system (SMILES) notation strings.

3. The system of claim 1, wherein the plurality of deep generative models is configured to generate molecular structures for a target of interest, or, result in a desirable omics signature.

4. The system of claim 1, wherein the plurality of deep generative models are configured to optimize the molecular structures based on objective functions of the individual plurality of deep generative models.

5. The system of claim 1, wherein the inter-model distance is the distance between molecular structures generated by a pair of generative models and intra-model distance is the distance between molecules generated by the same generative model.

6. The system of claim 5, wherein the deep neural network based molecular encoding model is configured to leverage the distances calculated to optimize the molecular structures for desired properties; wherein the desired properties are obtained by sampling the molecular structures that show small inter-model distances between the plurality of deep generative models of interest.

7. The system of claim 1, wherein the deep neural network based molecular encoding model is configured to obtain diverse molecular structures optimized for different properties of the plurality of deep generative models; wherein diverse molecular structures are obtained by sampling from overlapping regions occupied by molecular structures from the plurality of deep generative models of interest.

8. A method for generating customizable molecular structures for drug discovery, wherein the method comprises:
training a deep neural network based molecular encoding model, wherein a processor receives input datasets of drug-like molecules from private and public databases;
receiving input data relating to small molecules by a plurality of deep generative models; wherein the input data comprises data relating to desirable molecules and undesirable molecules;
generating molecular structures in accordance with the objective function of the generative model;
mapping similarities between the molecular structures generated from the plurality of deep generative models; wherein the molecular structures generated from the individual generative models are mapped on a n-dimensional latent space of the deep neural network based molecular encoding model;
computing intra-model and inter-model distances of the generated molecular structures; and
sampling the molecular structures generated from the plurality of deep generative models to obtain desired molecular structure.

9. The method of claim 8, wherein the method comprises representing the input datasets of drug-like molecules as simplified molecular-input line-entry system (SMILES) notation strings.

10. The method of claim 8, wherein the method comprises generating molecular structures by the plurality of deep generative models for a target of interest.

11. The method of claim 8, wherein the method comprises optimizing the molecular structures by the plurality of deep generative models based on objective functions of the individual plurality of deep generative models.

12. The method of claim 8, wherein the inter-model distance is the distance between molecular structures generated by a pair of generative models and intra-model distance is the distance between molecules generated by the same generative model.

13. The method of claim 12, wherein the method comprises leveraging the distances calculated by the deep neural network based molecular encoding model to optimize the molecular structures for desired properties; wherein the desired properties are obtained by sampling the molecular structures that show small inter-model distances between the plurality of deep generative models of interest.

14. The method of claim 8, wherein the method comprises obtaining diverse molecular structures optimized for different properties of the plurality of deep generative models; wherein diverse molecular structures are obtained by sampling from overlapping regions occupied by molecular structures from the plurality of deep generative models of interest.
